(19) United States
(12) Patent Application Publication
(10) Pub. No.: US 2006/0024346 A1
(43) Pub. Date: Feb. 2, 2006

(54) Stabilization of biologically active proteins with mixtures of polysaccharides and amino acid based compounds

(76) Inventors: Richard S. Brody, Worthington, OH (US); Sreedhara Alavattam, Columbus, OH (US); William M. Fountain, Columbus, OH (US); Randy L. Jones, Delaware, OH (US)

Correspondence Address: BATTELLE MEMORIAL INSTITUTE, 505 KING AVENUE, COLUMBUS, OH, 43201-2693, US

(21) Appl. No.: 10/901,784

(22) Filed: Jul. 29, 2004

(51) Int. Cl. A61K 38/19; A61K 38/21; A61K 39/395

(52) U.S. Cl. 424/423; 424/130.1; 424/85.1; 514/54; 514/12; 424/85.6

(57) ABSTRACT

The invention provides heat stable aqueous solutions or gels comprising a biologically active protein and a stabilizing effective amount of a mixture of a polysaccharide and an amino acid based compound. The invention also discloses stabilized solutions or gels suitable for use in an implantable drug delivery device at body temperature, and a device containing the stabilized solution or gels.

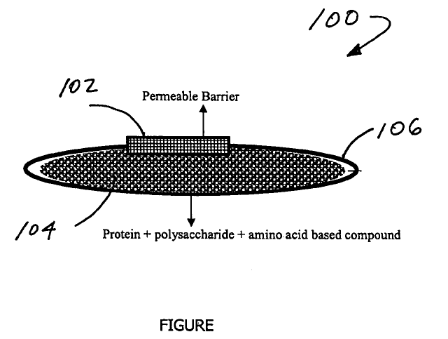

This application is related to and claims the benefit of U.S. application Ser. No. 10/012,667, filed Oct. 30, 2001; and WO 03/040398, designating the United States, the contents of which are incorporated herein by reference as if completely rewritten herein.

### FIELD OF THE INVENTION

The present invention relates to a heat stable aqueous solution or gel comprising a biologically active protein and an effective stabilizing mixture of a polysaccharide and amino acid based compound as well as heat stable solutions or gels suitable for use in a drug delivery device.

### BACKGROUND OF THE INVENTION

The commercial market for recombinant protein biopharmaceuticals is expanding rapidly as various biotechnology and pharmaceutical companies develop and test biologically active proteins. The emerging field of proteomics will likely provide protein targets useful for drug development, thereby enabling the market for recombinant protein biopharmaceuticals to continue its expansion.

Currently, proteins are utilized in a variety of diagnostic and therapeutic applications. For example, one protein used in a diagnostic application is the enzyme glucose oxidase, which is used in glucose assays. The hormone insulin is an example of a protein utilized in therapeutic applications. However, proteins are particularly sensitive to certain environmental conditions and may not be stable at elevated temperatures, including physiological temperature of 37° C., in non-optimal aqueous solvent systems, or in organic solvent systems. Protein stability may also be affected by pH and buffer conditions and exposure to shear forces or other physical forces.

The stability of a protein refers to both its conformational stability, which is reflected in the protein's three-dimensional structure, and its chemical stability, which refers to the chemical composition of the protein's constituent amino acids. Protein instability can result in a marked decrease or complete loss of a protein's biological activity. Deleterious stresses such as organic solvents, interfaces between organic and aqueous solvents, extremes of pH, high temperatures, and/or dehydration (drying) can affect both the conformational and chemical stability of a protein. Chemical instability can result from processes such as (a) deamidation of the amino acids residues asparagine or glutamine, (b) oxidation of cysteine or methionine amino acid residues, or (c) cleavage at any of the peptide amide linkages of the protein. Examples of conformational instability include aggregation (fibrillation), precipitation, and subunit dissociation. For reviews of protein stability see Arakawa et al., Advanced Drug Delivery Reviews, 46, 307-326 (2001) and Wang, International Journal of Pharmaceutics, 185, 129-188 (1999).

Because an inactive protein is useless, and in some cases deleterious, for most diagnostic and therapeutic applications, there is a need for a means by which proteins can be stabilized in solution at elevated temperatures (e.g. at and above room temperature, at body temperature or higher). This is particularly important for sustained release drug delivery systems where a therapeutic protein is incorporated into a device or polymer that is implanted or injected into a patient. During the time period when the protein is being released into the patient, which may last for months, it is critical that the protein remaining in the device or polymer retain its biological activity.

The typical method of administering therapeutic proteins to a patient or test subject is by means of needle-based injections. Currently, many pharmaceutical and drug delivery companies are seeking to develop alternative systems for the delivery of therapeutic proteins. These alternative systems are expected to require fewer dosings and to allow for more effective control over the rate of protein release in the body.

One alternative protein drug delivery system known in the art includes the formulation of the protein in a biodegradable, water insoluble, polymer matrix. The polymer (e.g., poly(lactic-co-glycolic acid)) can be formulated with protein as an injectable or respirable microparticle (Crotts and Park, Journal of Microencapsulation 15, 699-713, 1998). Alternately, the protein can be formulated in a temperature sensitive polymer that is liquid at room temperature but forms a solid gel at 37° C. after injection into a patient (Stratton et al., Journal of Pharmaceutical Sciences 86, 1006-1010, 1997). A third alternative is for the polymer to be dissolved in a non-toxic water miscible solvent that dissolves in plasma after injection leading to precipitation of the polymer (Yewey et al. Protein Delivery, Sanders and Hendren Eds., pp 93-117, Plenum Press, New York, 1997). In all cases, the polymer systems are developed for sustained release of protein over time; however, the stability of the protein during the release period is difficult to maintain and generally less than 50% of the total protein load can be delivered. Additionally, the delivery of the protein is not uniform, but rather occurs with a rapid initial burst which is followed by a much slower rate of sustained protein release (van de Weert et al., Pharmaceutical Research 17, 1159-1167, 2000).

A second type of known delivery system includes an implanted pump such as an osmotic pump (Kisker et al., Cancer Research 61, 7669-74, 2001; Kramer et al., Arch Biochem Biophysics, 368, 291-297, 1999; Stevenson et al. Handbook of Pharmaceutical Controlled Release Technology, D. L. Wise Ed., pp. 225-253, Marcel Dekker, New York, 2000). In this system, a protein solution or a suspension of protein in a water miscible organic solvent is continuously delivered to the patient or test subject through an orifice in the osmotic pump implant. A third type of delivery system is an implanted capsule with a semi-permeable membrane to control the rate of diffusion of the therapeutic protein from the capsule into the patient. All of the delivery systems discussed here require that the protein be stable in the device during the extended release periods.

It is known in the art that proteins can be stabilized in solution by the addition small hydrophilic molecules, such as disaccharides and amino acids, that stabilize the monomeric, correctly folded protein conformation. Disaccharides such as trehalose and sucrose and amino acids such as glycine, glutamate, or arginine are examples of compounds that are commonly used for stabilizing proteins (Timasheff, Advances in Protein Chemistry, 51, 355-432, 1998). Protein stabilization by small molecules, however, is not applicable for the polymer or capsule delivery systems. In both these cases, the small molecule stabilizer will diffuse out of the polymer or capsule at a faster rate than the much larger therapeutic protein, leaving the remaining protein without a stabilizer.

Inert proteins such as albumin and gelatin are well known to be protein stabilizers. Typically 0.1% to 1.0% of these proteins are added to a dilute solution of an active protein, such as an antibody, to keep the active protein from binding to the walls of the container or from aggregating.

There is a need to stabilize therapeutic proteins at 37° C. in drug delivery devices with stabilizers that will remain in the device while the protein diffuses out. The attachment of the protein to a solid support cannot be used for this application, as the immobilized protein is not likely to be released from the device and the biological activity of an immobilized protein is expected to be significantly lower than that of the free protein.

There are reports in the literature concerning the use of polysaccharide hydrogels and particles for drug delivery, as reviewed by Chen et al. (Carbohydrate Polymers 28, 69-76 (1995)). There is no disclosure in these reports of the ability of solutions of polysaccharides or polysaccharide composites to stabilize proteins under physiological conditions. Chen et al. (Biotechnology Letters 23, 331-333 (2001)) reported that soluble and insoluble starches stabilized the enzyme phytase at temperatures greater than 60° C. These researchers did not test combinations of starch with amino acid based compounds, especially for cases where starch was not a stabilizer by itself.

In related U.S. application Ser. No. 10/012,667 and WO 03/040398, high concentrations of high molecular weight polysaccharide gums are shown to be effective protein stabilizers at elevated temperatures. These stabilizers are very large molecules and can be retained in a capsule that will permit the release of the smaller therapeutic protein.

Despite the promising results obtained with polysaccharide gums, further improvement in protein stabilization is desirable for the application of this technology to sustained release drug delivery devices.

### BRIEF DESCRIPTION OF THE INVENTION

Broadly, in the present application discloses that the combination of polysaccharides with amino acid based compounds provides a much greater degree of protein stabilization than can be obtained with either component separately.

In the current invention, it is shown that improved stabilization of biologically active proteins can be obtained through the use of mixtures that contain a polysaccharide and one or more amino acid based compounds such as a protein, a poly(amino acid), an oligo(amino acid), and an amino acid. The polysaccharide component of the stabilizing mixture can be a polysaccharide gum or the hydrolyzed and reduced amylopectin fraction of starch. The amino acid based components can include a protein such as albumin or gelatin, a poly(amino acid) such as polyarginine, an oligo(amino acid) such as di-arginine, and an amino acid such as arginine.

The present invention is directed to stable aqueous solutions and gels of biologically active proteins wherein the active protein solutions and gels are stabilized by mixtures of polysaccharides and amino acid based compounds. The stable protein solutions and gels may be used in drug delivery systems and are protected against stresses such as high temperatures, oxidation, organic solvents, extremes of pH, drying, freezing, and agitation. Preferably, in the solutions and gels of the invention, the polysaccharides are not bound to the protein.

According to a preferred embodiment, the aqueous solutions or gels of the invention include at least one biologically active protein, wherein the protein may be an enzyme, antibody, hormone, growth factor, or cytokine and at least one polysaccharide for stabilizing the protein, wherein the polysaccharide, for example, may be gum arabic or amylopectin, and at least one amino acid based compound, wherein the amino acid based compound, for example, may be bovine serum albumin, bovine gelatin, polyarginine, oligo(arginine), or arginine.

Drug delivery systems compatible with the present invention include implanted subcutaneous delivery systems and intravenous drug delivery systems that can actively or passively deliver the biologically active proteins.

In one embodiment of the present invention, mixtures of high molecular weight polysaccharides and amino acid based compounds are used to stabilize therapeutic proteins delivered by means of implanted drug delivery devices such as a capsule, wherein the capsule includes a molecular weight cut-off membrane with uniform pore size. The mixture of the polysaccharide and amino acid based compound stabilizes the protein contained by the capsule and the release of the protein can be controlled by the membrane which is permeable to the therapeutic protein but impermeable to the higher molecular weight polysaccharide and amino acid based compounds. This embodiment, therefore, would not necessarily be compatible with small molecular weight stabilizers that would diffuse out of the capsule faster than the protein. The membrane retains the polysaccharide and the other stabilizers in the capsule and the capsule prevents the polysaccharide from swelling and decreasing in concentration.

A broad embodiment of the invention typically provides for a stabilized aqueous solution or gel that includes a biologically active protein; and a stabilizing effective amount of a polysaccharide; and an amino acid based compound. The biologically active protein is typically at least one enzyme, an antibody, a hormone, a growth factor, and a cytokine, and including mixtures thereof. Thus in some embodiments two or more polysaccharides and/or two or more amino acid based compounds may be used. In one preferred embodiment the active protein is human interferon-gamma. In other embodiments the polysaccharide is either a polysaccharide gum or a polysaccharide starch, and may be mixtures thereof. The polysaccharide gum is typically at least one of gum arabic, guar gum, xanthan gum, locust bean gum, tragacanth gum, gum karaya, gum ghatti, and hyaluronic acid, and including mixtures thereof. Preferably the polysaccharide gum is gum arabic.

In some embodiments when the polysaccharide is a polysaccharide starch it may be a waxy starch, a purified amylopectin, or mixtures thereof. In other embodiments when a waxy starch is used, the waxy starch may be a waxy corn starch, a waxy rice starch, a waxy wheat starch, a waxy potato starch, a waxy sorghum starch, or mixtures or two or more thereof. In one preferred embodiment, the polysaccharide starch has been hydrolyzed and reduced. Preferably the polysaccharide starch that has been hydrolyzed and reduced is potato amylopectin. In some embodiments the polysaccharide is present at from about 10% (w/v) to about 60% (w/v). In yet other embodiments the polysaccharide is present at from about 10% (w/v) to the polysaccharide's solubility limit. In some other embodiments, the polysaccharide starch that has been hydrolyzed and reduced is waxy corn starch. In yet other embodiments the polysaccharide is gum arabic, and the amino acid compound is porcine gelatin A. In other embodiments the polysaccharide is gum arabic, and the amino acid compound is bovine serum albumin. In further embodiments the polysaccharide is hydrolyzed waxy corn starch, and the amino acid compound is bovine serum albumin. Other useful embodiments are where the polysaccharide is hydrolyzed waxy corn starch, and the amino acid compound is bovine serum albumin and arginine; where the polysaccharide is hydrolyzed potato amylopectin, and the amino acid compound is bovine serum albumin.

In other embodiments, when a purified amylopectin is used, it is typically derived from cereal and/or tuber starches. In still other embodiments the purified amylopectin is selected from one or more of a corn starch, potato starch, rice starch, sorghum starch, wheat starch, and mixtures thereof.

In some embodiments the amino acid based compound is at least one of a protein, an amino acid, an amino acid oligomer, an amino acid polymer, or mixtures thereof. In other embodiments, a typical amino acid may be arginine, lysine, histidine, glutamic acid, aspartic acid, glycine, serine, proline, cysteine, methionine, asparagine, glutamine, threonine, or mixtures thereof. A preferred amino acid is arginine. In some other embodiments a typical amino acid oligomer may be a dimer, trimer, tetramer, or higher order oligomer that may be one or more of arginine, lysine, histidine, glutamic acid, aspartic acid, glycine, serine, proline, cysteine, methionine, asparagine, glutamine, threonine, or mixtures thereof. In yet other embodiments an amino acid polymer is typically a polyarginine, polylysine, polyhistidine, poly(glutamic acid), poly(aspartic acid), polyglycine, polyserine, polyproline, polycysteine, polymethionine, polyasparagine, polyglutamine, polythreonine, or mixtures thereof. In one embodiment the amino acid polymer is polyarginine. The protein may be a serum albumin or a gelatin that is derived from human, animal, or recombinant sources. One preferred serum albumin is bovine serum albumin. In yet other embodiments the stabilized aqueous gel is porcine gelatin A. In a yet further embodiment the amino acid compound is present at from about 1% (w/w) to about 10% (w/w). Typically, in a preferred embodiment, the amino acid compound is present at from about 1% (w/w) to the solubility limit of the amino acid compound.

Another embodiment provides for a stabilized aqueous solution or gel for use in an implantable drug delivery device including a pharmaceutically effective amount of a protein; and a stabilizing effective amount of a polysaccharide and an amino acid based compound.

A yet further embodiment provides for an implantable drug delivery device typically including a barrier permeable to a protein, a stabilized aqueous solution or gel within said barrier, wherein the stabilized aqueous solution or gel includes a pharmaceutically effective amount of the protein; and a stabilizing effective amount of a polysaccharide and an amino acid based compound.

### BRIEF DESCRIPTION OF THE DRAWING

The figure shows a schematic drawing of a typical implantable drug delivery device according to the invention

### DETAILED DESCRIPTION OF THE INVENTION AND BEST MODE

The present invention is directed to a heat stable aqueous solution or gel comprising an effective amount of a biologically active protein and a stabilizing effective amount of a mixture of a polysaccharide and amino acid based compounds. The invention is further directed to a heat stable aqueous solution or gel comprising an effective amount of a biologically active protein and a stabilizing effective amount of a mixture of a polysaccharide and amino acid based compounds, wherein the biologically active protein is selected from the group consisting of an enzyme, an antibody, a hormone, a growth factor, and a cytokine, wherein the polysaccharide is selected from the group consisting of polysaccharide gums, starches, and hydrolyzed starches, and wherein the amino acid based compounds are selected from the group consisting of proteins, amino acids, and poly(amino acids).

Another embodiment of the invention relates to a heat stable solution or gel comprising a pharmaceutically effective amount of a biologically active protein and a stabilizing effective amount of a mixture of polysaccharide and amino acid based compound, wherein the stabilized solution or gel is contained in an implantable drug delivery device.

As used herein the term “biologically active protein” includes proteins and polypeptides that are administered to patients as the active drug substance for prevention of or treatment of a disease or condition as well as proteins and polypeptides that are used for diagnostic purposes, such as enzymes used in diagnostic tests or in in vitro assays as well as proteins that are administered to a patient to prevent a disease such as a vaccine. Contemplated for use in the compositions of the invention, but not limited to, pharmaceutically effective amounts of therapeutic proteins and polypeptides such as enzymes, e.g., glucocerebrosidase, adenosine deaminase; antibodies, e.g., Herceptin® (trastuzumab), Orthoclone OKT®3 (muromonab-CD3); hormones, e.g., insulin and human growth hormone (HGH); growth factors, e.g., fibroblast growth factor (FGF), nerve growth factor (NGF), human growth hormone releasing factor (HGHRF); cytokines, e.g., leukemia inhibitory factor (LIF), granulocytemacrophage-colony stimulating factor (GM-CSF), interleukin-6 (IL-6), interleukin-11 (IL-11), interleukin-9 (IL-9), oncostatin-M (OSM), ciliaryneurotrophic factor (CNTF), and interferon-γ; vaccines, e.g. HA protein flu vaccine, Hepatitis B surface antigen vaccine, and Pneumococcal protein vaccine.

The term “pharmaceutically effective amount” refers to that amount of a therapeutic protein having a therapeutically relevant effect on a disease or condition to be treated. A therapeutically relevant effect relieves to some extent one or more symptoms of a disease or condition in a patient or returns to normal either partially or completely one or more physiological or biochemical parameters associated with or causative of the disease or condition. Specific details of the dosage of a particular active protein drug may be found in the drug labeling, i.e., the package insert (see 21 CFR § 201.56 & 201.57) approved by the United States Food and Drug Administration.

The polysaccharides described in this invention are typically natural products extracted from plant and tree sources such as polysaccharide gums, e.g., gum arabic, guar gum, xanthan gum, locust bean gum, tragacanth gum, gum karaya, gum ghatti, hyaluronic acid; waxy polysaccharide starches, e.g., waxy corn starch, waxy rice starch, waxy wheat starch, waxy potato starch, and waxy sorghum starch; and purified amylopectin polysaccharides, e.g., corn amylopectin, rice amylopectin, wheat amylopectin, potato amylopectin, and sorghum amylopectin. In some embodiments derivatives of the natural substances or equivalents synthesized by industrial or pharmaceutical processes may also be used.

Gum arabic is produced by the Acacia senegal tree. The gum Arabic used in the solutions of the invention is a highly branched molecule with a main chain of (1 to 3) linked β-D-galactopyranosyl units having multiple oligo-galactopyranosyl side chains attached via (1 to 6) linkages. Both the main chain and the side chains have multiple linkages to other sugars consisting mainly of α-L arabinofuranosyl, α-L-rhamnopyranosyl, β-D glucuronopyranosyl, and 4-O-methyl-β-D-glucuronopyranosyl units. Gum arabic also consists of about 1% protein, which is heavily glycosylated. The molecular weight of gum arabic is over 300,000 daltons. Other high molecular weight polysaccharide gums, such as guar gum, xanthan gum, locust bean gum, tragacanth gum, gum karaya, and gum ghatti have been shown to stabilize model proteins with efficacies similar to that of gum arabic (see related U.S. application Ser. No. 10/012,667 and WO 03/040398). It is therefore reasonable that these gums can be used in the stabilizing mixture in place of gum arabic. The present disclosure provides data that hyaluronic acid is another polysaccharide that stabilizes a model protein at elevated temperatures in the presence and absence of additional amino acid stabilizers.

The amino acid based compounds used in the stabilizing mixtures can be proteins, e.g. albumin and gelatin; hydrophilic amino acids, e.g. arginine, lysine, histidine, glutamic acid, aspartic acid, glycine, serine, proline, cysteine, methionine, asparagine, glutamine, threonine; oligo(amino acids), e.g. di-arginine, tri-arginine, and tetra-arginine and polyaminoacids, e.g., polyarginine, polylysine, polyhistidine, poly(glutamic acid), poly(aspartic acid), polyglycine, polyserine, polyproline, polycysteine, polymethionine, polyasparagine, polyglutamine, and polythreonine.

The proteins described in the examples are bovine serum albumin and porcine gelatin A, but it is expected that albumins and gelatins from other sources, such as human proteins isolated from blood or recombinant human proteins, will have the same stabilizing effect. Both the serum albumins (MW about 66,000 daltons) and the gelatins (about MW 50,000-100,000 daltons) are significantly larger than therapeutic cytokines such as recombinant interferon-γ (MW about 17,000). Poly(amino acids) can be synthesized with size distributions that are also much larger than that for cytokines.

As used herein, the Polysaccharide Solubility Limit is the concentration of polysaccharide obtained after an aqueous buffer, typically a phosphate buffered saline (PBS), is slowly added to a solid polysaccharide, with thorough mixing, until all of the solid material has either dissolved or has hydrated to form a gel. Depending on the polysaccharide used, the solubility limit can be in the vicinity of about 10% or can be higher than about 60%. Physiological condition as pertained to this invention is typically human body temperature under normal conditions, that is, 37° C. a neutral pH of around 7±1, and a physiological concentration of saline (0.9%).

Related U.S. application Ser. No. 10/012,667 and WO 03/040398 show that high concentrations of gum arabic stabilize multiple proteins to incubation at elevated temperature and at 37° C. In the case of the therapeutic protein interferon-γ, gum arabic prepared by dialysis and lyophilization was shown to stabilize the immunological activity of the protein, as determined by ELISA (enzyme linked immuno assay). In the current invention, it was found that the anti-viral activity of interferon-γ, in contrast to the immunological activity, was poorly stabilized by the standard gum arabic preparation. Composites of gum arabic and gelatin A, on the other hand, were effective stabilizers of the anti-viral activity of interferon-γ (Table 1).

It was found that heated gum arabic is an effective stabilizer of the antiviral activity of interferon-γ (Table 2). While not wishing to be bound by theory, this is presumably due to inactivation of oxidase and peroxidase enzymes that are know to be associated with gum arabic and which are inactivated by heat treatment (Glicksman and Schachat, Industrial Gums, R. L. Whistler Ed., pp. 213-298, Academic Press, New York, 1959). Table 2 also shows that the addition serum albumin to heated gum arabic further enhances its ability to stabilize interferon-γ. The ability of heated gum arabic to stabilize interferon-γ is highly dependent on the gum arabic concentration, as seen in Table 3.

The waxy corn starch and potato amypectin described in this invention are both composed almost entirely of amylopectin, which is a highly branched structure consisting of chains of (1 to 4) linked α-D-glucopyranosyl units joined together via α-D-(1 to 6) linkages. The molecular weight of amylopectin is greater than 50 million daltons. The size of the amylopectin chains can be reduced by acid hydrolysis, resulting in a more highly soluble preparation of lower viscosity and less tendency to gel at high concentrations, and the terminal reducing sugar at the end of each chain can be reduced by the action of sodium borohydride (U.S. Pat. Nos. 3,523,938 and 4,016,354). Waxy corn starch and potato amylopectin have been hydrolyzed by acid and reduced by the method described in the reference. These two materials are called hydrolyzed waxy corn starch and hydrolyzed amylopectin respectively.

Hydrolyzed waxy corn starch and hydrolyzed potato amylopectin are poor stabilizers for interferon-γ. In the presence of protein (serum albumin), amino acid compounds (arginine and polyarginine), or combinations of these compounds, however, the corn starch preparation exhibits greatly improved stabilizing properties (Table 4). In the presence of protein, the potato amylopectin preparation was also shown to exhibit greatly enhanced ability to stabilize protein (Table 5).

The ability of mixtures of polysaccharides and amino acid based compounds to stabilize the enzymes lactate dehydrogenase and glucose-6-phosphate dehydrogenase is shown in Tables 6 and 7. Mixtures of hydrolyzed corn starch with either bovine serum albumin (BSA), arginine, or arginine+BSA significantly stabilized the lactate dehydrogenase towards incubation at elevated temperature (Table 6). In contrast, hydrolyzed corn starch, BSA, or arginine by themselves offered no significant stabilization for this enzyme.

Glucose-6-phosphate dehydrogenase is also stabilized by a mixture that contains hydrolyzed corn starch, BSA, and arginine (Table 7). In this case, however, the separate components or mixtures of two components do not stabilize this enzyme significantly.

The polysaccharides used herein are typically used at concentrations that are near or at the upper limit of the solubility of the particular polysaccharide in aqueous solutions. Gum arabic, hydrolyzed waxy corn starch, and hydrolyzed potato amylopectin have exceptional solubility in aqueous solution and formulations containing 60% of these polysaccharides have been made. These formulations, while viscous, can be transferred with a positive displacement pipette. The addition of amino acid based compounds to the concentrated polysaccharide solutions increases their viscosities and makes them more gel-like. This is especially apparent in the case of 56% corn starch+3.7% BSA+6% arginine, which forms a thick, sticky, formulation. The combination of 50% heated gum arabic+10% BSA, in contrast, is a clear syrup that can be transferred by a positive displacement pipette. This formulation provides the best stabilization of interferon-γ found in this study, resulting in solutions that retain approximately 70% of their anti-viral activity after 1 month at 37° C.

Hyaluronic acid, which has a history of use in humans, was found to be an effective stabilizer of the enzyme activity of chymotrypsin at elevated temperatures (Table 8). This polysaccharide gum appears to behave similarly to the stabilizing gums described in related U.S. patent application Ser. No. 10/012,667 and WO 03/040398.

The stability of the cytokine interferon-α was tested with several of the polysaccharide/amino acid compound formulations at 37° C., as described in Example 16. Unlike the results with interferon-γ, none of the formulations stabilized interferon-α. While both interferon-γ and interferon-α are both cytokines, they have different physical properties. Interferon-γ has a high isoelectric point and is positively charged at neutral pH while interferon-α has a low isoelectric point and is negatively charged at neutral pH. This suggests that at least some of the polysaccharide/amino acid compound formulations may only stabilize cytokines that have a net positive charge under neutral, physiological conditions.

Polysaccharides are hydrogels that can absorb many times their weight of water. Therefore, it is preferable to restrict the tendency of the polysaccharides to swell in order to maintain the high polysaccharide concentrations that are essential for protein stabilization (Table 3). The high gum concentration can be maintained by enclosing the gels in a capsule with a molecular membrane that is permeable to the protein but impermeable to the higher molecular weight polysaccharide, protein, or polyamino acid. The capsules can be implanted into a patient or test subject for the controlled release of stabilized protein over extended periods. Over time, the protein is steadily released from the capsule, thus decreasing the concentration of protein inside the capsule while the concentration of the stabilizing gum within the capsule remains constant.

In various embodiments, the compositions of the present invention are utilized for the stabilization of proteins during membrane-controlled release from capsules or other devices implanted into a patient or test subject. In this case, the delivery device is designed to prevent the polysaccharide from swelling so that the stabilizing effects of high polysaccharide concentrations are maintained inside the capsule. Since it is unnecessary for the polysaccharides and amino acid based compounds described herein to bind to biologically active proteins to effect stabilization, biologically active proteins can be released from the solution or gel by diffusion. Additionally, the polymeric properties of polysaccharides provide another mechanism for stabilization by restricting a protein's molecular mobility.

The Figure illustrates a typical embodiment for an implantable drug delivery device 100 according to the invention including a permeable barrier 102 permeable to a protein, a stabilized material (such as a stabilized aqueous solution or gel) 104 within said permeable barrier 102, wherein the stabilized material includes a pharmaceutically effective amount of the protein; and a stabilizing effective amount of a polysaccharide and an amino acid based compound. The permeable barrier 102 encloses as least a portion of the stabilized material 104 as shown in the Figure with the remainder of the enclosing formed by a nonpermeable capsule material 106. In some embodiments the permeable barrier 102 will completely enclose the stabilized material 104 (not shown in the Figure) as will be appreciated by those skilled in the art.

The stabilized protein solutions and gels of the invention may contain minor amounts (from about 0.5% to about 5.0% w/v) of auxiliaries and/or excipients, such as N-acetyl-dl-tryptophan, caprylate, acetate, citrate, glucose and electrolytes, such as the chlorides, phosphates and bicarbonates of sodium, potassium, calcium and magnesium. They can furthermore contain: acids, bases or buffer substances for adjusting the pH, salts, sugars or polyhydric alcohols for isotonicity and adjustment, preservatives, such as benzyl alcohol or chlorobutanol, and antioxidants, such as sulphites, acetylcysteine, Vitamin E or ascorbic acid.

Suitable tonicity adjustment agents may be, for instance, physiologically acceptable inorganic chlorides, e.g. sodium chloride; sugars such as dextrose; lactose; mannitol; sorbitol and the like. Preservatives suitable for physiological administration may be, for instance, esters of parahydroxybenzoic acid (e.g., methyl, ethyl, propyl and butyl esters, or mixtures of them), chlorocresol and the like.

According to the present invention, a preferred method for stabilizing a therapeutic protein in a drug delivery system comprises the steps of (a) providing a biologically active protein as an aqueous solution; and (b) adding a polysaccharide and amino acid based compounds to the active protein. Typically a subsequent step may be (c) adding the solution or gel to a capsule that contains a molecular membrane. The membrane is typically fabricated from silica or a polymer and has pore sizes, which permit the membrane to be permeable to the protein but relatively or substantially impermeable to the higher molecular weight polysaccharide and amino acid based compounds. The stabilized therapeutic protein is typically provided in pharmaceutically effective amounts.

A drug delivery system typically comprises pharmaceutically effective amounts of therapeutic protein stabilized by polysaccharides and amino acid based compounds, wherein the stabilized therapeutic protein is provided in a pharmaceutically effective carrier. Typical examples of carriers are mentioned herein.

The following examples are illustrative rather than limiting and are not intended to limit the scope of the embodiments or claims of the invention in any way.

### EXAMPLE 1

This example illustrates source of materials used herein and any preliminary preparation of the materials. Recombinant human Interferon-γ was purchased from PBL Biomedical Laboratories and Shandong GeneLeuk Biopharmaceutical Co., Ltd. The protein from both suppliers showed a single protein band by gel electrophoresis at about 17,000 daltons and had the same anti-viral biological activity per mg of protein. Gum arabic, chymotrypsin, BSA, porcine gelatin A (300 bloom; 50,000-100,000 daltons), waxy corn starch, potato amylopectin, L-arginine (arg), L-lysine, poly-L-arginine 5,000-15,000 daltons (polyarg), human umbilical cord hyaluronic acid (MW about 4,000,000 daltons), Streptococcal hyaluronic acid (MW about 750,000 daltons), lactate dehydrogenase and glucose-6-phosphate dehydrogenase were obtained from Sigma. Eagle's Minimum Essential Medium (EMEM), Fetal Bovine Serum (FBS), and murine encephalomycarditis virus (EMCV) were obtained from the ATCC (American Type Culture Collection; Manassas, Va., USA). The Hetastarch (hydroxyethyl starch) used In these studies was Hespan® which was obtained as a 6% solution from Edwards Biomedical Supply. Hespan® was dialyzed against water and lyophilized before use. MTS was obtained from Promega (Cell Titer 96 AQueous one solution cell proliferation assay).

### EXAMPLE 2

This example illustrates the preparation of gum arabic. Gum arabic (100 g) was dissolved in deionized water (1 L) and the pH of the solution was adjusted to 7.4 by the addition of 4 M sodium hydroxide. After the solution was centrifuged at 30,100×g for 10 minutes, the supernatant was filtered through an 11 μm nylon screen filter and then concentrated to approximately 300 mL on a Millipore Prep/Scale TFF-6 Tangential Flow Filter with a molecular weight cut off of 10,000 daltons. The volume of the concentrate was adjusted to 1 liter by the addition of deionized water and the process of concentration and reconstitution was repeated for a total of five cycles. After the final concentration, the 300 mL concentrate was transferred to a beaker. The filter apparatus was washed with about 100 mL aliquots of deionized water that were combined with the 300 mL concentrate until the volume of the concentrate was increase to 1 liter. The pH was then adjusted to 7.4 with 4 M sodium hydroxide and the sample was filtered through a 0.22 μm Millipak 200 in-line filter using a peristaltic pump. The filtrate was divided into two approximately 500 mL aliquots that were frozen and lyophilized. The lyophilized product was then ground using a mortar and pestle and the resulting powder was stored at 4° C.

### EXAMPLE 3

This example illustrates the preparation of heated gum arabic. Gum arabic that was dialyzed and lyophilized (Example 1) was dissolved in deionized water as a 10% (w/w) solution. The sample was heated with vigorous magnetic stirring in a boiling water bath for 45 minutes. The solution was then cooled and the pH adjusted to 7.4 with 0.1 M sodium hydroxide. The sample was then lyophilized and the resulting solid was ground with a mortar and pestle and stored at 4° C.

Gum arabic tested positive for peroxidase enzymes before heating and tested negative for the enzymes after heat treatment, as determined by a calorimetric assay using 3,3′, 5,5′-Tetramethylbenzidine (TMB) liquid substrate for ELISA (Sigma Chemical Company).

### EXAMPLE 4

This example illustrates the preparation of hydrolyzed waxy corn starch. Waxy corn starch (80 g) was combined with 0.01 M hydrochloric acid (400 mL) in a 500 mL 3-neck round bottom flask with an overhead stirrer and a reflux condenser. The sample was heated with overhead stirring for approximately three hours at 87.5° C., at which time the overhead stirrer was removed and an egg shaped magnetic stirrer was added to the sample. The heating at 87.5° C. was continued until the sample had been heated for a total of 24 hours (the 24 hour period began when the sample was heated sufficiently to form a paste, which occurred between about 70° C. and 75° C.). The sample was then cooled, the pH adjusted to 7.0 with a saturated aqueous solution of sodium bicarbonate, and the sample transferred to a large crystallizing dish and diluted with 400 mL deionized water. Sodium borohydride (8 g) was then slowly added, with magnetic stirring, to the sample and the stirring was continued for 5 minutes after all of the sodium borohydrode had been added. Unreacted borohydride was then decomposed by the addition of glacial acetic acid until further addition of acetic acid produced no additional effervescence. The sample was then adjusted to pH 7 with saturated sodium bicarbonate and autoclaved for 20 minutes at 121° C. The autoclaved sample was then centrifuged at 10,000×g for 10 minutes and filtered through a 0.22 μm filter. The sample was then dialyzed at 4° C. against deionized water in dialysis tubing with a 50,000 dalton molecular weight cut for a total of two days. The dialysis water was changed twice daily. The solution was then lyophilized, redissolved in water to make a 25% (w/w) starch solution, and adjusted to pH 7.4 with 0.1 M sodium hydroxide. Finally, the sample was diluted to 5%, filtered through a 0.22 μm filter, lyophilized and the resulting solid was ground with a mortar and pestle and stored at 4° C.

### EXAMPLE 5

This example illustrates the preparation of hydrolyzed potato amylopectin. Potato amylopectin (10 g) was combined with 50 mL deionized water in a three neck flask that had a reflux condenser. The sample was heated in a 60° C. water bath until the amylopection dissolved. Hydrochloric acid (0.5 mL of a 1 M solution) was then added and the sample was stirred with an egg shaped magnetic stirrer as it was heated to 87.5° C. Stirring was continued at 87.5° C. for 24 hours. The sample was then cooled, the pH adjusted to 7.0 with a saturated aqueous solution of sodium bicarbonate and the sample transferred to a large crystallizing dish and diluted with 50 mL deionized water. Sodium borohydride (1 g) was then slowly added, with magnetic stirring, to the sample and the stirring was continued for 5 minutes after all of the sodium borohydride had been added. Unreacted borohydride was then decomposed by the addition of glacial acetic acid until further addition of acetic acid produced no additional effervescence. The sample was then adjusted to pH 7 with saturated sodium bicarbonate and autoclaved for 20 minutes at 121° C. The autoclaved sample was then centrifuged at 10,000×g for 10 minutes and filtered through a 0.22 μm filter. The sample was then dialyzed at 4° C. against deionized water in dialysis tubing with a 50,000 dalton molecular weight cut for a total of two days. The dialysis water was changed twice daily. The solution was then lyophilized, redissolved in water to make a 25% (w/w) starch solution, and adjusted to pH 7.4 with 0.1 M sodium hydroxide. Finally, the sample was diluted to 5%, filtered through a 0.22 μm filter, lyophilized, and the resulting solid was ground with a mortar and pestle and stored at 4° C.

### EXAMPLE 6

This example illustrates the preparation of gum arabic/gelatin A mixtures. Gum arabic/gelatin A mixtures were prepared by mixing 5% solutions of gum arabic (Example 1) in deionized water with 5% solutions of porcine gelatin A (300 bloom) at selected weight ratios. The pH of the gelatin solutions were adjusted to pH 7.4 prior to combining with the gum arabic. The composite samples were heated to 60° C., mixed well, and then shell frozen and lyophilized.

### EXAMPLE 7

This example illustrates the preparation and workup of stabilized interferon-γ samples. Samples containing interferon-γ and either gum arabic, gum arabic/Gelatin A, waxy corn starch or potato amylopectin were made by the addition of solutions of interferon-γ (typically 0.1 mg/mL) in PBS/0.5% sodium azide to the solid polysaccharide or polysaccharide mixture in different weight ratios. Samples containing interferon-γ, a polysaccharide, and BSA, arginine, polyarginine, or combinations of these additives were made by first diluting the interferon in a solution of amino acid based compounds made in PBS/0.5% sodium azide. This solution was then added to the solid polysaccharide. Typically, the interferon-γ solutions were added to 30-50 mg of solid stabilizer to make the desired formulations. All compositions were expressed as weight percentages.

The samples were incubated in a humidified container at 37° C. in a closed polypropylene tube with a solid insert to reduce the volume (tube volume with insert about 0.1 mL). After incubation, the insert was removed (tube volume without insert about 1 mL) and the samples were diluted 20 fold by the addition of EMEM assay media with 1% FBS. The samples were then mixed with a toothpick until the stabilizer was either dispersed or dissolved, and then vigorously mixed with a vortex mixer. Additional dilutions were then made in the same media to obtain concentrations in the ng/mL range suitable for the assay.

### EXAMPLE 8

This example illustrates the determination of antiviral activity of interferon-γ samples. The anti-viral activity for interferon-γ was determined via a virus-induced cytopathic effect inhibition assay as described by Meager (Journal of Immunological Methods 261, 21-36, 2002) and Khaber et al. (Journal of Interferon and Cytokine Research, 16, 31-33, 1996). Vero cells were plated in a 96 well tissue culture plate using EMEM culture medium with 10% FBS by the addition to each well of 0.1 mL of a solution containing 6×10⁴ cells/mL. The cells were incubated overnight at 37° C. in a 5% CO₂ atmosphere to obtain a monolayer at a confluence of about 75-80%. After the medium was decanted and the wells washed twice with EMEM, inteferon-γ samples were added in culture medium and the cells incubated at 37° C. for 7-8 hours at 37° C. in 5% CO₂. Cells were then challenged with 0.1 mL of EMCV, suitably diluted at a determined plaque forming units/mL, in culture medium containing 1% FBS. The plate was then incubated overnight, or until development of extensive cytopathlogy (80-90% cytopathic effect) in unprotected cells). Quantitative estimation of the cytopathic effect inhibition was determined by adding MTS solution (3-(4,5 dimethylthiazol-2-yl)-5-(3-carboxymethoxyphenyl)-2-(4-sulfophenyl)-2H-tetrazolium) to each well containing either the samples or the standard curve in 0.1 mL of EMEM culture medium with 1% FBS. The plate was incubated for 1 to 3 hrs at 37° C., and the absorbencies were recorded at 490 nm using an ELISA plate reader. The range used for the assay standard curve was 0.03 to 15 ng/mL interferon-γ.

### EXAMPLE 9

This example illustrates the stabilization of interferon-γ antiviral activity at 37° C. in gum arabic/gelatin A formulations. Interferon-γ was incubated at 37° C. in PBS solutions that contained gum arabic, gelatin A, and in gum arabic/gelatin A mixtures. Table 1 shows the antiviral activity that remained after 2 and 4 weeks in these solutions.

TABLE 1
Stabilization of Interferon-γ Antiviral Activity at 37°
in Gum Arabic/Gelatin A Formulations^{a}

| | ANTIVIRAL ACTIVITY | |
|---|---|---|
| FORMULATION | 2 Weeks | 4 Weeks |
| PBS | <3% | ND |
| PBS + 50% Gum Arabic | 6 ± 1% | 0.2 ± 0.1% |
| PBS + 33% Gelatin A | <1%^{b} | ND |
| PBS + 33% (Gum Arabic/Gelatin A, 4:1) | 50 ± 20% | 40 ± 10% |
| PBS + 33% (Gum Arabic/Gelatin A, 3:2) | 50 ± 2% | 60 ± 40% |

^{a}The data in this table contains a single significant figure. Entries with error bars (± %) were the average of two separate experiments, each run with duplicate samples, ± the deviation from the mean.
ND = no data taken at this time point.

### EXAMPLE 10

This example illustrates the stabilization of interferon-γ antiviral activity at 37° C. in heated gum arabic/BSA formulations. Interferon-γ was incubated at 37° C. in PBS solutions that contained Tween, Hetastarch, BSA, heated gum arabic, and heated gum arabic+BSA. Table 2 shows the antiviral activity that remained after 2, 4, and 8 weeks in these solutions.

TABLE 2
Stabilization of Interferon-γ Antiviral Activity at 37° in Heated
Gum Arabic/BSA Formulations^{a}

| | ANTIVIRAL ACTIVITY | | |
|---|---|---|---|
| FORMULATION | 2 Weeks | 4 Weeks | 8 Weeks |
| PBS | <3% | ND | ND |
| PBS + 0.5% Tween 20 | <3% | ND | ND |
| PBS + 50% Hetastarch | <1% | ND | ND |
| PBS + 10% BSA | ND | <1% | ND |
| PBS + 50% Heated Gum Arabic | 70 ± 30% | 40 ± 10% | 9 ± 6% |
| PBS + 50% Heated Gum Arabic + | 70% | 70% | 20% |
| 10% BSA | | | |
| PBS + 50% Heated Gum Arabic + | 70% | 70% | 4% |
| 5% BSA | | | |

^{a}The data in this table contains a single significant figure. Entries without error bars were the average of duplicate samples in a single experiment. Entries with error bars were the average of at least four separate experiments, each run with duplicate samples, ±1 standard deviation.
ND = no data taken at this time point.

### EXAMPLE 11

This example illustrates the effect of heated gum arabic concentrations on the stabilization of interferon-γ antiviral activity at 37° C. Interferon-γ was incubated at 37° C. in PBS solutions that contained heated gum arabic at different concentrations. Table 3 shows the antiviral activity that remained after 2 weeks in these solutions.

TABLE 3
Effect of Heated Gum Arabic Concentration on the Stabilization
of Interferon-γ Antiviral Activity at 37°^{a}

| | 2 Week Antiviral |
|---|---|
| Formulation | Activity |
| PBS + 33% Heated Gum | 1% |
| Arabic | |
| PBS + 40% Heated Gum | 20% |
| Arabic | |
| PBS + 45% Heated Gum | 50% |
| Arabic | |
| PBS + 50% Heated Gum | 60% |
| Arabic | |
| PBS + 55% Heated Gum | 70% |
| Arabic | |

^{a}The data in this table contains a single significant figure. Entries were the average of duplicate samples in a single experiment.

### EXAMPLE 12

This example illustrates the stabilization of interferon-γ antiviral activity at 37° C. in hydrolyzed waxy corn starch formulations. Interferon-γ was incubated at 37° C. in PBS solutions that contained waxy corn starch, BSA, arginine, polyarginine, and combinations of these materials. Table 4 shows the antiviral activity that remains after 1, 2, and 4 weeks in these solutions.

TABLE 4
Stabilization of Interferon-γ Antiviral Activity at 37° in
Hydrolyzed Waxy Corn Starch Formulations^{a}

| | ANTIVIRAL ACTIVITY | | |
|---|---|---|---|
| FORMULATIONS | 1 Week | 2 Weeks | 4 Weeks |
| PBS | <1% | ND | ND |
| PBS + 14.7% Arg | 3% | ND | <1% |
| PBS + 9% BSA | ND | <1% | <1% |
| PBS + 8.5% BSA + 13.5% Arg | <1% | ND | ND |
| PBS + 9% Polyarginine^{b} | ND | <1% | ND |
| PBS + 60% Hydrolyzed Waxy | 2% | 7 ± 7% | 2% |
| Corn Starch | | | |
| PBS + 57% Hydrolyzed Waxy | 80% | 60 | 9% |
| Corn Starch + 4% BSA | | | |
| PBS + 56% Hydrolyzed Waxy | 50% | ND | 1% |
| Corn Starch + 6.5% Arg | | | |
| PBS + 56% Hydrolyzed Waxy | 100 ± 50% | ND | 40 ± 10% |
| Corn Starch + 6% Arg + | | | |
| 3.7% BSA | | | |
| PBS + 57% Hydrolyzed Waxy | ND | 10% | ND |
| Corn Starch + 4% Polyarginine^{b} | | | |
| PBS + 56% Hydrolyzed Waxy | ND | 40% | ND |
| Corn Starch + 4% Polyarginine + | | | |
| 4% BSA^{b} | | | |

^{a}The data in this table contains a single significant figure. Entries without error bars were the average of duplicate samples in a single experiment. Entries with error bars were the average of two separate experiments, each run with duplicate samples, ± the deviation from the mean;
ND = no data taken at this time point.
^{b}The weight percentages in the polyarginine solutions were only estimates. Polyarginine solutions were made by combining a weighed polyarginine sample and a known mass of PBS. A measured volume of this solution was then added to the sample. Unlike the cases with arginine and BSA, the densities of the polyarginine solutions were not measured, but rather the densities of similarly prepared BSA solutions were used in these calculations.

Experiments were also performed to determine the ability of solutions that contained approximately 50% hydrolyzed corn starch to stabilize inteferon-γ. 50% hydrolyzed corn starch, like 60% hydrolyzed corn starch, did not by itself stabilize interferon-γ significantly. Samples that contained about 50% corn starch and BSA, arginine, or arginine+BSA stabilized interferon-γ, but to a lesser extent than analogous formulations containing about 60% corn starch.

### EXAMPLE 13

This example illustrates the stabilization of interferon-γ antiviral activity at 37° C. in hydrolyzed potato amylopectin formulations. Interferon-γ was incubated at 37° C. in PBS solutions that contained hydrolyzed potato amylopectin and BSA. Table 5 shows the antiviral activity that remained after two weeks in these solutions.

TABLE 5
Stabilization of Interferon-γ Antiviral Activity at 37° in
Hydrolyzed Potato Amylopectin Formulations^{a}

| | ANTIVIRAL ACTIVITY |
|---|---|
| FORMULATIONS | AFTER TWO WEEKS |
| 60% Hydrolyzed Potato Amylopectin | 8% |
| 57% Hydrolyzed Potato Amylopectin + 4% | 80% |
| BSA | |

^{a}The data in this table contains a single significant figure. Entries were the average of duplicate samples in a single experiment.

### EXAMPLE 14

This example illustrates the stabilization of lactate dehydrogenase and glucose-6-phosphate activities at 60° C. in hydrolyzed corn starch formulations. The abilities of mixtures of hydrolyzed corn starch and amino acid compounds to stabilize the enzymes glucose-6-phosphate dehydrogenase and lactate dehydrogenase is shown in Tables 6 and 7. Lactate dehydrogenase was assayed by the method of Lovell and Winzor (Biochemistry 13, pp 3527 -3531, 1974). Glucose-6-phosphate dehydrogenase was assayed by the method of Sola-Penna and Meyer-Fernandes (Arch. Biochem. Biophys, 360, pp 10-14 (1998).

TABLE 6
Stabilization of Enzymatic Activity of Lactate
Dehydrogenase (LDH) at Elevated Temperatures
in Hydrolyzed Waxy Corn Starch Formulations^{a}.

| | Incubation | % Activity |
|---|---|---|
| Formulation | Conditions | Remaining |
| PBS | 60° C., 10 min | 1 ± 1% |
| PBS + 14.7% ARG | 60° C., 10 min | 1% |
| PBS + 9% BSA | 60° C., 10 min | 0% |
| PBS + 60% Hydrolyzed Waxy Corn | 60° C., 10 min | 3 ± 0% |
| Starch | | |
| PBS + 56% Hydrolyzed Waxy Corn | 60° C., 10 min | 78 ± 1% |
| Starch + 6.5% ARG | | |
| PBS + 57% Hydrolyzed Waxy Corn | 60° C., 10 min | 72 ± 8% |
| Starch + 4% BSA | | |
| PBS + 56% Hydrolyzed Waxy Corn | 60° C., 10 min | 104 ± 0% |
| Starch + 6% ARG + 3.7% BSA | | |

^{a}Entries with error bars represent the average of two separate experiments ± the deviation from the mean. Entries without error bars represent the results of a single experiment.

TABLE 7
Stabilization of Enzymatic Activity of Glucose-6-Phosphate
Dehydrogenase at Elevated Temperatures in Hydrolyzed
Waxy Corn Starch Formulations^{a}.

| | Incubation | % Activity |
|---|---|---|
| Formulation | Conditions | Remaining |
| PBS | 60° C., 10 min | 1 ± 1% |
| PBS + 14.7% ARG | 60° C., 10 min | 3% |
| PBS + 9% BSA | 60° C., 10 min | 0% |
| PBS + 60% Hydrolyzed Waxy Corn Starch | 60° C., 10 min | 1 ± 0% |
| PBS + 56% Hydrolyzed Waxy Corn | 60° C., 10 min | 2 ± 0% |
| Starch + 6.5% ARG | | |
| PBS + 57% Hydrolyzed Waxy Corn | 60° C., 10 min | 8 ± 1% |
| Starch + 4% BSA | | |
| PBS + 56% Hydrolyzed Waxy Corn | 60° C., 10 min | 52 ± 3% |
| Starch + 6% ARG + 3.7% BSA | | |

^{a}Entries with error bars represent the average of two separate experiments ± the deviation from the mean. Entries without error bars represent the results of a single experiment.

### EXAMPLE 15

This example illustrates the stabilization of chymotrypsin by hyaluronic acid (HA). Hyaluronic acid from human umbilical cord and hyaluronic acid from Streptococcus species were tested for their abilities to stabilize the enzyme chymotrypsin at elevated temperatures. Aliquots that contained 0.05 mL of chymotrypsin (1 mg/mL) in PBS or chymotrypsin (1 mg/mL)+BSA (5%) in PBS were added to 17.8 mg samples of hyaluronic acid. The samples were mixed until all the hyaluronic acid was hydrated, forming a viscous solution. The mixtures were heated at 60° C. for 7.5 min in a water bath. A similar solution was prepared and was used as room temperature control without heating.

The hyaluronic acid samples with chymotrypsin were assayed as follows: PBS (0.95 mL) was added to the hyaluronic acid/chymotrypsin samples, and the diluted material was homogenized for 1 min on ice. Aliquots of 0.05 mL of the above solution were further diluted with 0.95 mL PBS. Samples of 0.05 mL of the final dilution were used to assay for chymotrypsin activity using N-benzoyl tyrosine ethyl ester (BTEE) as the substrate.

As can be seen in Table 8, chymotrypsin incubated with hyaluronic acid from Streptococcus species retained all activity upon heating at 60° C. for 7.5 min, conditions under which chymotrypsin loses almost all its activity in PBS alone. Chymotrypsin heated in the presence of 5% BSA retained about 16% of its activity, but in the presence of a combination of hyaluronic acid and BSA, chymotrypsin retained all its activity upon heating. Similar results were obtained with hyaluronic acid from human umbilical cord.

TABLE 8
Stabilization of chymotrypsin by hyaluronic acid at 60° C.^{a}

| | PERCENT | |
|---|---|---|
| | CHYMOTRYPSIN | |
| | ACTIVITY | |
| SAMPLES | ROOM TEMP | 60° C. |
| Chymotrypsin | 100 | 1 |
| Chymotrypsin + 26% hyaluronic acid | 100 | 107 |
| Chymotrypsin + 5% BSA | 100 | 16 |
| Chymotrypsin + 5% BSA + 26% hyaluronic | 100 | 121 |
| acid, | | |

^{a}The entries in this table were the average of duplicate samples in a single experiment.

### EXAMPLE 16

This example illustrates and compares stabilization of interferon-α antiviral activity at 37° C. in polysaccharide/amino acid based compound formulations. Interferon-α was incubated at 37° C. in the presence of PBS, Gum Arabic (50%), Gum Arabic/Gelatin A, 4:1 (33%), Gum Arabic/Gelatin A, 3:2 (33%), hydrolyzed waxy corn starch (60%), and hydrolyzed waxy corn starch (56%)+1 M arginine (6.5%). The antiviral activity of interferon-α was monitored via the same virus-induced cytopathic effect inhibition assay that was used for interferon-γ and described in Example 8. In all cases, the stability of interferon-α after one to eight weeks in PBS was equal to or greater than the stability of interferon-α in any of the polysaccharides or polysaccharides+amino acid based compounds tested.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

We claim:

1. A stabilized aqueous solution or gel comprising:
A. a biologically active protein; and
B. a stabilizing effective amount of,
a. a polysaccharide; and
b. an amino acid based compound.

2. The stabilized aqueous solution or gel according to claim 1, wherein the biologically active protein is selected from the group consisting of an enzyme, an antibody, a hormone, a growth factor, a cytokine, and mixtures thereof.

3. The stabilized aqueous solution or gel according to claim 1, wherein two or more polysaccharides and/or two or more amino acid based compounds are used.

4. The stabilized aqueous solution or gel according to claim 2, wherein the active protein is human interferon-gamma.

5. The stabilized aqueous solution or gel according to claim 1, wherein the polysaccharide is selected from the group consisting of a polysaccharide gum, a polysaccharide starch, and mixtures thereof.

6. The stabilized aqueous solution or gel according to claim 5, wherein the polysaccharide gum is selected from the group consisting of gum arabic, guar gum, xanthan gum, locust bean gum, tragacanth gum, gum karaya, gum ghatti, hyaluronic acid, and mixtures thereof.

7. The stabilized aqueous solution or gel according to claim 5, wherein the polysaccharide starch is selected from the group consisting of waxy starches, purified amylopectins, and mixtures thereof.

8. The stabilized aqueous solution or gel according to claim 7, wherein the waxy starches are selected from the group consisting of waxy corn starch, waxy rice starch, waxy wheat starch, waxy potato starch, waxy sorghum starch, and mixtures thereof.

9. The stabilized aqueous solution or gel according to claim 7, wherein the purified amylopectin is derived from cereal or tuber starches.

10. The stabilized aqueous solution or gel according to claim 7, wherein the purified amylopectin is selected from the group consisting of corn starch, potato starch, rice starch, sorghum starch, wheat starch, and mixtures thereof.

11. The stabilized aqueous solution or gel according to claim 7, wherein the polysaccharide starch has been hydrolyzed and reduced.

12. The stabilized aqueous solution or gel according to claim 1, wherein the polysaccharide is present at from about 10% (w/v) to the polysaccharide's solubility limit.

13. The stabilized aqueous solution or gel according to claim 1, wherein the amino acid based compound is selected from the group consisting of a protein, an amino acid, an amino acid oligomer, an amino acid polymer, and mixtures thereof.

14. The stabilized aqueous solution or gel according to claim 13, wherein the protein comprises a serum albumin and a gelatin derived from human, animal, or recombinant sources.

15. The stabilized aqueous solution or gel according to claim 13 wherein the amino acid is selected from the group consisting of arginine, lysine, histidine, glutamic acid, aspartic acid, glycine, serine, proline, cysteine, methionine, asparagine, glutamine, threonine and mixtures thereof.

16. The stabilized aqueous solution or gel according to claim 13, wherein the amino acid oligomer comprises a dimer, trimer, tetramer, or higher order oligomer selected from the group consisting of arginine, lysine, histidine, glutamic acid, aspartic acid, glycine, serine, proline, cysteine, methionine, asparagine, glutamine, threonine, and mixtures thereof.

17. The stabilized aqueous solution or gel according to claim 13, wherein the amino acid polymer is selected from the group consisting of polyarginine, polylysine, polyhistidine, poly(glutamic acid), poly(aspartic acid), polyglycine, polyserine, polyproline, polycysteine, polymethionine, polyasparagine, polyglutamine, polythreonine, and mixtures thereof.

18. The stabilized aqueous solution or gel according to claim 1, wherein the polysaccharide comprises gum arabic, and the amino acid compound comprises porcine gelatin A.

19. The stabilized aqueous solution or gel according to claim 1, wherein the polysaccharide comprises gum arabic, and the amino acid compound comprises bovine serum albumin.

20. The stabilized aqueous solution or gel according to claim 1, wherein the polysaccharide comprises hydrolyzed waxy corn starch, and the amino acid compound comprises bovine serum albumin.

21. The stabilized aqueous solution or gel according to claim 1, wherein the polysaccharide comprises hydrolyzed waxy corn starch, and the amino acid compound comprises bovine serum albumin and arginine.

22. The stabilized aqueous solution or gel according to claim 1, wherein the polysaccharide comprises hydrolyzed potato amylopectin, and the amino acid compound comprises bovine serum albumin.

23. The stabilized aqueous solution or gel according to claim 1, wherein the amino acid compound is present at from about 1% (w/w) to about 10% (w/w).

24. The stabilized aqueous solution or gel according to claim 1, wherein the amino acid compound is present at from about 1% (w/w) to the solubility limit of the amino acid compound in the polysaccharide solution.

25. A stabilized aqueous solution or gel for use in an implantable drug delivery device comprising:
a pharmaceutically effective amount of a protein; and
a stabilizing effective amount of a polysaccharide and an amino acid based compound.

26. An implantable drug delivery device comprising:
a barrier permeable to a protein,
a stabilized aqueous solution or gel within said barrier, wherein said stabilized aqueous solution or gel comprises,
a pharmaceutically effective amount of said protein; and
a stabilizing effective amount of a polysaccharide and an amino acid based compound.
